(19) United States
(12) Patent Application Publication
(10) Pub. No.: US 2020/0170612 A1
(43) Pub. Date: Jun. 4, 2020

(54) Tissue Mimicking Materials

(71) Applicant: TECHNOLOGICAL UNIVERSITY DUBLIN, Dublin (IE)

(72) Inventors: Jacinta Browne, Dublin (IE); Andrew Fagan, Dublin (IE); Andrea Doyle, Dublin (IE)

(21) Appl. No.: 16/640,027

(22) PCT Filed: Aug. 17, 2018
(86) PCT No.: PCT/EP2018/072369

§ 371 Date: Feb. 18, 2020

(30) Foreign Application Priority Data
Aug. 17, 2017 (GB) 1713229.1

(51) Int. Cl. A61B 8/08; A61B 5/055; G09B 23/28

(52) U.S. Cl. CPC A61B 8/08; G09B 23/286; A61B 8/485; A61B 5/055

(57) ABSTRACT

A biopsy training device (100) is described. The device comprises a skin mimicking layer (102) and a fibroglandular tissue mimicking portion (104). The fibroglandular tissue mimicking portion comprises one or more lesion mimicking portions and is removably coupled to the skin mimicking layer. A tissue mimicking material, a mixture for forming a tissue mimicking material and a method of manufacturing a tissue mimicking material are also described.

The present invention provides a mixture for forming a tissue mimicking material, a tissue mimicking material, a biopsy training device, and a method of making a tissue mimicking material.

Tissue mimicking materials (TMMs) may be used to provide a realistic simulation of tissue or other anatomical structures. TMMs may be used in devices for training medical practitioners in imaging (e.g. ultrasound imaging) and performing biopsies or other surgical procedures. For example, TMMs used in imagining training may form “phantoms” adapted to create an anatomically realistic representation of the desired human or animal anatomy when imaged using an appropriate imaging technique. Such a phantom can also be used in measuring and calibrating imaging equipment and its effect on tissue. TMMs used for biopsy or other surgical training may be more simplistic, but should nonetheless provide a visual and haptic representation of real anatomy. This allows the user to develop the coordination skills required to perform a biopsy procedure without the need to practice on real tissue.

Prior art TMM materials provide varying levels of anthropomorphic realism and so the quality of training they can provide is limited. Prior art devices may be overly simplistic in design and not replicate either the morphology or ultrasonic properties of the tissue being mimicked. This may result in unrealistic ultrasound images which do not adequately challenge a trainee's ability to perform ultrasound scanning. For example, lesions contained within the training devices may be very easy to find. Furthermore, once lesions are detected, prior art imaging phantoms do not have characteristics that accurately replicate tumours in vivo, and so this component of the training may be completely absent. Prior art training devices may be overly rigid and do not provide adequate haptic feedback to trainees. As a result of inadequate TMM training devices, medical training may be done on the job, rather than in safe controlled conditions.

It is an object of the present application to provide TMMs having an improved anthropomorphic nature and thus provide an improved realistic representation of the desired anatomy. By providing an improved anatomical representation the resulting TMMs can provide training devices which aid a trainee's ability to detect important pathologies, as well as improving patient care and reducing misdiagnoses.

In one aspect, the present invention provides a mixture for forming a tissue mimicking material, the mixture comprising: a) a gelling agent; b) water; c) glycerol; d) one or more scattering particles; and e) silicone emulsion.

By including silicone emulsion in the TMM, improved realism from an ultrasound point of view can be achieved. This therefore may help to replicate the real situation in vivo. The use of silicone emulsion helps to improve the visual appearance of the images by providing improved darkness levels while also providing the desired levels of attenuation. The silicone emulsion added to the TMM increases the viscosity and frictional forces opposing the transition of a sound wave through the TMM. This may therefore allow the attenuation of sound waves to be controlled to the desired level to simulate real tissue. Prior art TMMs use an increased concentration of particles to increase the attenuation of sound waves. This however also increases the brightness of the image from within the TMM making it difficult to control the brightness and the attenuation independently. The TMM of this application solves this problem by the use of silicone emulsion to control the attenuation without affecting the brightness of the image.

Optionally, the silicone emulsion may comprise 3% to 10% of the total weight of the tissue mimicking material. This range has been found to provide realistic simulation of tissue.

Optionally, the silicone emulsion comprises between 3.5% and 4.3%, and preferably 3.92%, of the total weight of the tissue mimicking material and preferably the water comprises between 74% and 75%, and further preferably 74.41%, of the total weight of the tissue mimicking material. This may produce a tissue mimicking material suited to mimicking glandular tissue, and particularly glandular tissue found in the breast.

Optionally, the silicone emulsion comprises between 6.7% and 10%, and preferably 8.37%, of the total weight of the tissue mimicking material and the water preferably comprises between 75% and 76%, and further preferably 75.34%, of the total weight of the tissue mimicking material. This may produce a tissue mimicking material suited to mimicking malignant lesions and particularly malignant lesions found in the breast.

Optionally, the gelling agent comprises between 1.5% and 5% of the total weight of the tissue mimicking material, and preferably comprises 3% of the total weight of the tissue mimicking material. This may provide a realistic representation of various types of tissue.

Optionally, the gelling agent comprises a mixture of Konjac and Carrageen. The Carrageen may be Carrageen-Iota or Carrageen-Kappa.

Advantageously, using Carrageen-iota provides a TMM that is thixotropic in nature. This can be advantageous for mimicking the haptic nature of tissue such as prostate tissue. It can mimic the acoustic and mechanical properties to provide corresponding haptic feedback and compliance of soft tissues under needle puncture. The TMM can be optimised to achieve a range of mechanical properties, for example young's modulus values. This is an important physical property to mimic as tissue stiffness is usually indicative of disease. As such this material has a future application in the development of ultrasound elastography and magnetic resonance imaging elastography training and quality assurance phantoms. As the TMM exhibits a thixotropic behaviour when punctured, it exhibits “self-healing” properties. For example, when the TMM is punctured by a biopsy needle, the needle tracks or striations produced by the needle fade as the material “self-heals” through this thixotropic behaviour. This improves the usefulness and longevity of the device.

In prior art training biopsy phantoms, where there is a TMM with speed of sound and attenuation coefficients which represent the average of different tissues types (e.g. liver, kidney, muscle and arterial), the contrast of the TMMs in the ultrasound image is not clinically relevant and therefore does not provide a suitable training platform. Furthermore, the nature of the solid gel TMM used in these TMMs means that biopsy devices have a very short shelf-life after the first needle punctures as well as providing an unsuitable training tool as the track indicates the position of the targets which need to be biopsied.

Optionally, the ratio of the weight of Konjac to Carrageen in the tissue mimicking material is in the range of 10:90 to 90:10, and wherein the ratio is preferably any one of: 50:50, 60:40, 40:60 or 65:35. This may provide the desired level of elasticity and haptic properties according to the type of tissue being mimicked.

Optionally, the one or more scattering particles are chosen from any one or more of: i) Silicon carbide particles; or ii) Aluminium Oxide particles. This may increase the ultrasound backscatter coefficient of the TMM as desired.

Optionally, the mixture may further comprise a preservative, wherein the preservative may preferably be Benzalkonium chloride or a mixture of sulphanic acid and oxalic acid. Alternatively the preservative may be or comprise mould and/or mildew. This may help reduce bacterial invasion of the TMM.

Optionally, the mixture may further comprise potassium chloride or sodium chloride. Potassium chloride is preferable when Carrageen-Kappa is used as a gelling agent; Sodium chloride is preferable when Carrageen-Iota is used as a gelling agent. This may help stop the strands of the carrageen powder used to form the gelling agent from being hydrophobic and not mixing with the water i.e. facilitate wetting and therefore gelling.

Advantageously, in embodiments, components of the TMM can be manipulated to achieve acoustic and mechanical characteristics of a range of tissues in the body, speed of sound, attenuation coefficient and backscatter (tissue relevant contrast).

In another aspect, the present invention provides a tissue mimicking material comprising: a first portion formed from a mixture comprising: Konjac, Carrageen, and water; and a second portion formed from a mixture comprising Konjac, Carrageen, and water, wherein the ratio of Konjac to Carrageen in the first portion is different from the ratio of Konjac to Carrageen in the second portion.

By providing different portions of the TMM with different ratios of Konjac to Carrageen, the hepatic feedback provided by the TMM may be improved. This may give more realistic pliability of the TMM and so give a more realistic simulation of the hand-to-eye coordination required to perform a biopsy procedure.

Optionally, the ratio of the weight of Konjac to Carrageen in the first and/or the second portion may be in a range of 10:90 to 90:10. This may provide a realistic level of haptic feedback for a number of different types of human (or animal) tissue.

Optionally, the ratio of the weight of Konjac to Carrageen in the first portion may be chosen from any one of: 40:60, 50:50, 60:40 and 65:35, and the ratio of the weight of Konjac to Carrageen in the second portion may be chosen from another one of: 40:60, 50:50, 60:40 and 65:35. These ratios may provide the desired level of haptic feedback corresponding to different types of tissue.

Optionally, the total amount of Konjac and Carrageen in the first portion and/or the total amount of Konjac and Carrageen in the second portion may be in the range of 1.5% to 5% by weight of the respective first or second portions. This may help provide a realistic level of haptic feedback.

Optionally, the first portion and the second portion may be chosen from any of the following portions having different ratios of Konjac and Carrageen to each other:

a) a fat layer mimicking portion comprising the Konjac, Carrageen and water, and further comprising one or more scattering particles, oil and a surfactant, wherein the weight of the Konjac and Carrageen is in a ratio of 40:60;
b) a glandular tissue mimicking portion comprising the Konjac, Carrageen and water, and further comprising one or more scattering particles, glycerol and silicone emulsion, wherein the weight of the Konjac and Carrageen is in a ratio of 60:40;
c) a Cooper Ligament mimicking portion comprising the Konjac, Carrageen and water, and further comprising one or more scattering particles and glycerol, wherein the weight of the Konjac and Carrageen is in a ratio of 50:50;
d) a pectoral muscle mimicking portion comprising the Konjac, Carrageen and water, and further comprising one or more scattering particles and glycerol, wherein the weight of the Konjac and Carrageen is in a ratio of 50:50;
e) a malignant lesion mimicking portion comprising the Konjac, Carrageen and water and further comprising one or more scattering particles, glycerol and silicone emulsion, wherein the weight of the Konjac and Carrageen is in a ratio of 40:60;
f) a benign lesion mimicking portion comprising the Konjac, Carrageen and water and further comprising one or more scattering particles and glycerol, wherein the weight of the Konjac and Carrageen is in a ratio of 65:35;
g) an anechoic lesion mimicking portion comprising the Konjac, Carrageen, and water and further comprising glycerol, wherein the weight of the Konjac and Carrageen is in a ratio of 40:60; and
h) a hyperechoic lesion mimicking portion comprising the Konjac, Carrageen and water and further comprising one or more scattering particles and glycerol, wherein the weight of the Konjac and Carrageen is in a ratio of 40:60.

This may allow different portions of the TMM to be tailored to mimic respective different types of tissue.

Optionally, the silicone emulsion may form between 3.5% and 4.3% by weight, and preferably 3.92% by weight, of the glandular tissue mimicking portion, and/or the silicone emulsion may form between 6.7% and 10% by weight, and preferably 8.37% by weight, of the malignant lesion mimicking portion. These values have been found to help provide a realistic representation of real tissue.

Optionally, the first portion may comprise the fat layer mimicking portion; the second portion comprises the glandular tissue mimicking portion; the tissue mimicking material may further comprise: the Cooper Ligament mimicking portion; the pectoral muscle mimicking portion; and any one or more of: at least one malignant lesion mimicking portion; at least one benign lesion mimicking portion; at least one anechoic lesion mimicking portion; and at least one hyperechoic lesion mimicking portion. This combination of portions of tissue mimicking material may allow a realistic representation of the breast to be provided.

The quantities (e.g. percentage by weight) and relative ratios of components given above and throughout the description and claims relate to the final TMM rather than quantities of components added to a mixture from which the TMM is formed. The relative quantities and ratios of components added to create the TMM forming mixture may vary from those in the resulting TMM.

In another aspect, the present invention provides a biopsy training device, comprising: a skin mimicking layer; and a fibroglandular tissue mimicking portion comprising one or more lesion mimicking portions, wherein the fibroglandular tissue mimicking portion is removably coupled to the skin mimicking layer.

By providing a fibroglandular tissue mimicking portion that is removable it may be removed and replaced after a number of uses of the TMM. During biopsy training, parts of the tissue mimicking material are removed and so the material has a limited useful lifespan (e.g. 10-15 uses) and it may otherwise be disposed of. The skin mimicking layer does not however become damaged at the same rate. The use of the removable fibroglandular portion allows the biopsy training device to be “refilled” once all of the training lesions have been removed or damaged. This provides an improvement over the prior art where an entire TMM material may have to be disposed of when all of the lesions have been damaged or removed, but the skin mimicking layer is less damaged and would otherwise be adequate for continued use.

Optionally, the biopsy training device may further comprise a base member arranged to removably couple to the skin mimicking layer, wherein the base member and the skin mimicking layer may form a housing when coupled in which the fibroglandular tissue mimicking portion is received. The housing allows the fibroglandular mimicking portion to be removed and replaced by disconnecting the base member.

Optionally, the fibroglandular tissue mimicking portion may be coupled to an inside wall of the skin mimicking layer by a coupling liquid comprising water, glycerol and Benzalkonium chloride (BC). This may help couple the glandular tissue mimicking portion and skin mimicking portion to provide ultrasound transmission between them.

In another aspect, the present invention provides a method of manufacturing a tissue mimicking material, comprising any one of the following steps: a) adding one or more scattering particles to water; b) adding gelling agent to the mixture formed in step a), wherein the mixture is mixed at a first rate during the addition of the gelling agent; c) heating the mixture formed in part b), wherein during the heating the mixture is mixed at a second mixing rate, the second mixing rate being slower than the first; and d) forming the mixture formed in part c) into a desired shape.

By providing a higher mixing rate during the addition of the gelling agent the formation of clumps of gelling agent can be avoided. A second lower mixing rate helps to ensure homogeneous mixing of the metallic particles while minimising the creation of air bubbles. The method of the present invention therefore provides a homogeneous TMM with consistent acoustic properties.

Optionally, the first mixing rate may be between 200 and 260 rpm and the second mixing rate may be between 110 and 160 rpm. These ranges of mixing rates may provide improved mixing of the gelling agent and a homogeneous resulting TMM.

Optionally, the mixture may be at ambient temperature during the addition of the gelling agent in step b).

Optionally, the heating of step c) may comprise heating to a temperature of 90 to 100° C. Heating the mixture allows gelation of the gelling agent to take place.

Optionally, the heating in step c) may have a duration of 1 hour. By heating the mixture for this period of time a suitable matrix structure for the scattering particles to be uniformly distributed through is provided.

Optionally, the method may further comprise adding glycerol to the resulting mixture formed in step c).

Optionally, the method may further comprise heating the glycerol before it is added, the glycerol preferably being heated to a temperature in the range of 70 to 90° C. By heating the glycerol congealing of the TMM may be avoided.

Optionally, the method may further comprise adding Benzalkonium chloride to the glycerol. The Benzalkonium chloride may act as a preservative to reduce bacteria invasion. In other embodiments, a different preservative may be added. Mould and/or mildew may instead be added to the TMM as a preservative.

Optionally, step a) may further comprise adding silicone emulsion to the water, the silicone emulsion may be added before the addition of the scattering particles. As discussed in relation to the first aspect, this may allow the ultrasound attenuation to be controlled.

Optionally, the method may further comprise adding oil and surfactant to the resulting mixture formed in step c).

Optionally, the oil and surfactant may be heated before being added, the oil and surfactant preferably being heated to a temperature of greater than 80° C.

Optionally, the method may further comprise mixing the mixture resulting from the addition of the oil and surfactant at a third mixing rate.

Optionally, the third mixing rate may be between 200 rpm and 150 rpm, and may preferably comprise a first mixing period of 200 rpm and a second mixing period of 150 rpm.

Optionally, the mixture may be heated during addition of the oil and surfactant and/or during mixing at the third mixing rate.

Optionally, the mixture may be mixed at a fourth mixing rate after mixing at the third mixing rate, wherein the mixture may be allowed to cool while mixing at the fourth mixing rate. This may help to produce a more homogeneous TMM.

Optionally, the fourth mixing rate may be 250 to 320 rpm and preferably about 290 rpm. This mixing rate has been found to improve the homogeneity of the TMM.

Optionally, forming the mixture into the desired shape may comprise shaping the mixture in a mould.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the mixture, TMM or training device may have corresponding features definable with respect to the method(s), and vice versa, and these embodiments are specifically envisaged.

Embodiments of the invention will now be described, by way of example only, with reference to the accompanying drawings, in which:

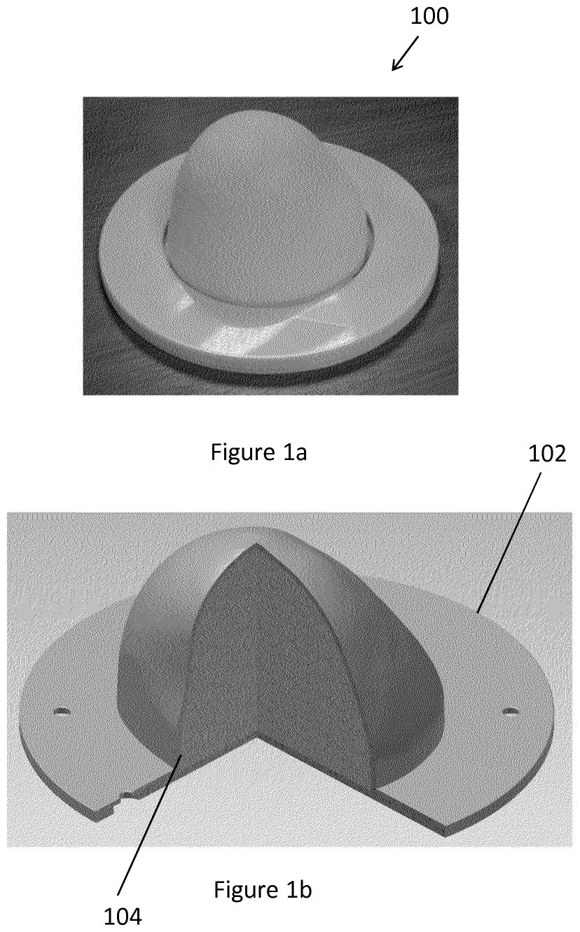

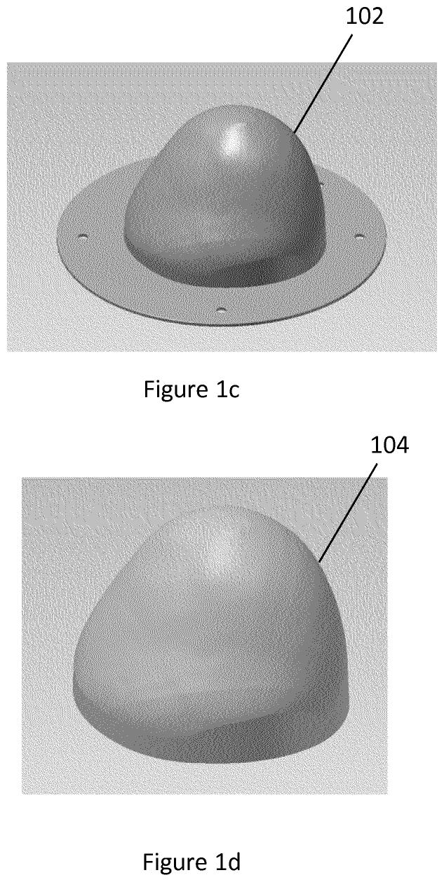

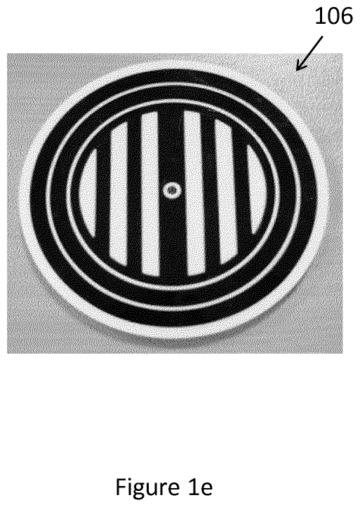

FIG. 1a shows a perspective view of a biopsy training device according to an embodiment;

FIG. 1b shows a cut away schematic view of part of the biopsy training device shown in FIG. 1a;

FIG. 1c shows a skin mimicking layer of the biopsy training device shown in FIG. 1a;

FIG. 1d shows a fibroglandular tissue mimicking portion of the biopsy training device shown in FIG. 1a;

FIG. 1e shows a base member of the biopsy training device shown in FIG. 1a; and

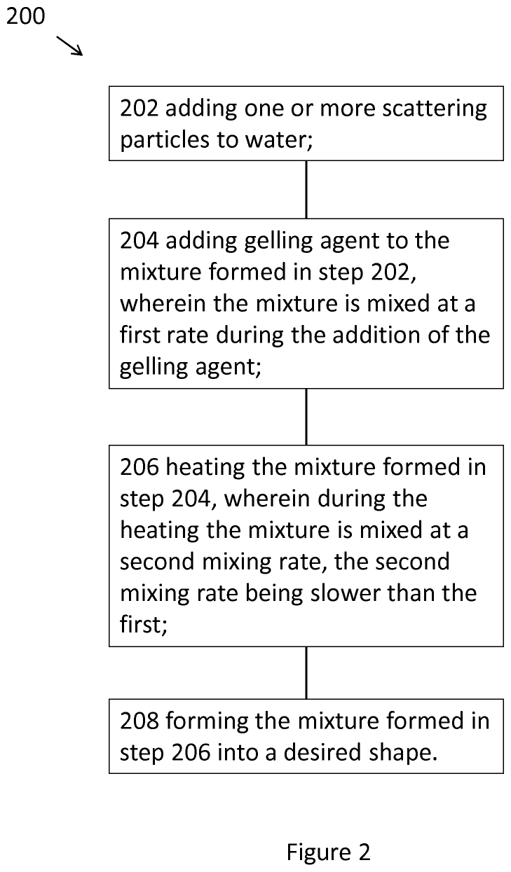

FIG. 2 shows a method of manufacturing a tissue mimicking material according to an embodiment.

### Tissue Mimicking Material Forming Mixture

In one aspect, the present disclosure provides a mixture for forming a tissue mimicking material (TMM). The mixture may be initially produced in a liquid state before being solidified to form a TMM as is known in the art. In some embodiments, two or more mixtures having different materials or different relative quantities of certain materials may be used to make a single TMM (e.g. by forming separate layers or areas within the TMM as descried in the following section).

The resulting TMM formed using the mixture may be used in an ultrasound phantom (e.g. for B-mode ultrasound imaging) suitable for the simulation of human or animal tissue. The TMM may be formed into a phantom by being sealed in a suitable container or housing as is known in the art. In order to provide a TMM giving a realistic representation of actual tissue the same ranges of speeds of sound, attenuation coefficients and backscatter coefficients should be recreated. The resulting TMM may be used to mimic a range of different types of tissue or other anatomical structures as required. In one embodiment, the resulting TMM may be used to form part of a TMM for the simulation of the breast including pathologies such as malignant lesions and fibro-adenoma (this is described in more detail in the following section). In other embodiments, the materials and quantities used in the TMM forming mixture may be chosen to mimic other tissue as required.

A mixture for forming a TMM (generally referred to as the “mixture”) according to one aspect of the present disclosure generally comprises:
a) a gelling agent;
b) water;
c) glycerol;
d) one or more scattering particles; and
e) silicone emulsion.

The inclusion of silicone emulsion in the resulting TMM improves the visual appearance of the TMM when it is imaged using ultrasound. This is achieved by altering the speed of sound within the TMM. The addition of silicone emulsion has been found to provide a suitable level of darkness in an ultrasound image of the TMM while also providing a suitable level of attenuation. By the addition of silicone emulsion, the TMM of the present invention provides improved anthropomorphic properties compared to TMMs of the prior art. This may provide improved and more challenging training for medical practitioners.

In some embodiments, the TMM may include further components in addition to those listed above. In yet other embodiments, the TMM may consist essentially of only the components listed above (e.g. only components that do not materially affect the visual appearance of an ultrasound image, or the haptic properties, of the TMM, e,g. a preservative, may also be present). In yet other embodiments, the TMM consists of only the components listed above (e.g. no other components are present).

The proportion of the TMM made up of silicone emulsion may be tailored according to the type of tissue that is to be mimicked. The ratio of silicone emulsion to the other components may at least partly determine the attenuation of an ultrasound beam incident on the TMM and the speed of sound in the material. In some embodiments, the silicone emulsion may comprise 3% to 10% of the total weight of the TMM. Levels of silicone elusion in this range may advantageously allow a realistic image of various types of human (or animal) tissue to be generated.

In some embodiments, the amount of silicone emulsion (or other components) included in the mixture may be varied independently to the other materials included (e.g. the other materials may remain in the same relative quantities to each other). In some embodiments, the amount of one or more of the other materials may be adjusted to account for an increased or decreased amount of silicone emulsion (or changes in other component). In one such embodiment, the amount of water included in the mixture may be altered to account for a change in amount of silicone emulsion.

In some embodiments, the silicone emulsion comprises 3.92% of the total weight of the TMM. In this embodiment, the silicone emulsion may vary in amount between 3.5% and 4.3% of the total weight of the TMM while still providing the same advantageous properties. The amount of water may comprise 74.41% of the total weight of the TMM. In this embodiment, the water may vary in amount between 74% and 75% of the total weight of the TMM while still providing the same advantageous properties. This embodiment may be particularly suited to mimicking glandular tissue, which typically has an ultrasound attenuation of 2 dB/cm/MHz and speed of sound of 1500 m/s. Tests carried out on a TMM made using the mixture of this embodiment have been found to result in an attenuation of 1.87±0.038 dB/cm/MHz and speed of sound of 1531.83±0.71 m/s (see the examples of TMMs provided later in the application).

In other embodiments, the silicone emulsion comprises 8.37% of the total weight of the tissue mimicking material. In this embodiment, the silicone emulsion may vary in amount between 6.7% and 10% of the total weight of the TMM while still providing the same advantageous properties. The amount of water may comprise 75.34% of the total weight of the tissue mimicking material. In this embodiment, the water may vary in amount between 75% and 76% of the total weight of the TMM while still providing the same advantageous properties. This embodiment may be particularly suited to mimicking malignant lesions, which typically have an ultrasound attenuation of 1 dB/cm/MHz (at 7 MHZ) and a speed of sound of 1550 m/s. Tests carried out on a TMM made using the mixture of this embodiment have been found to result in an attenuation of 0.99±0.025 dB/cm/MHz (at 7 MHZ) and speed of sound of 1492.49±0.41 m/s (see the examples of TMMs provided later in the application).

The proportion of gelling agent forming the mixture may be chosen in order to provide a resulting TMM having the desired consistency, pliability and haptic properties. In some embodiments, the gelling agent may comprise between 1.5% and 5% of the total weight of the TMM. Preferably, the gelling agent may comprise 3% of the total weight of the tissue mimicking material. These values have been found to provide realistic representations of various types of human or animal tissue.

Any suitable gelling agent known in the art may be used in the mixture. In one embodiment, the gelling agent may comprise a mixture of Konjac and Carrageen. The Carrageen may be Carrageen-Kappa or Carrageen-Iota. The ratio of Konjac to Carrageen may be varied in order to provide a suitable TMM elasticity and desired haptic properties. In some embodiments, the ratio of the weight of the Konjac to Carrageen may be in the range of 10:90 to 90:10. This has been found to provide a realistic level of haptic feedback for a number of different types of human (or animal) tissue. The ratio of Konjac to Carrageen may be varied independently of the other components of the mixture to achieve the desired properties.

In some embodiments, the ratio may be chosen from any one of: 50:50, 60:40, 40:60 or 65:35 according to the type of tissue to be mimicked by the TMM. As discussed in the following section, a ratio of 50:50 may be suitable for mimicking Cooper's ligament tissue (or other ligament tissue) or pectoral muscle (or other muscle tissue); a ratio of 60:40 may be suitable for mimicking glandular tissue; a ratio of 40:60 may be suitable for mimicking a fat layer, fibro-adenoma, malignant lesions, anechoic lesions or hyperechoic lesions; and a ratio of 65:35 may be suitable for mimicking benign lesions. The skilled person will understand however that these ratios are only provided as advantageous examples and each ratio could be used to simulate other types of tissue. In some embodiments, any suitable ratios may be used in order to provide the desired TMM properties. In yet other examples, alternative gelling agents may be used (such as agar, for example).

The mixture of the described embodiment may comprise one or more scatting particles arranged to increase the ultrasound backscatter coefficient of the resulting TMM. The scatting particles may be formed by solid particles added to the mixture. A uniform suspension of scattering particles may be formed once the mixture has solidified to form the TMM.

The one or more scattering particles may be chosen from Silicon carbide particles or Aluminium Oxide particles. The size of the individual scattering particles and the relative amount included in the mixture may be chosen according to the desired ultrasound scattering properties required. In some embodiments, a mixture of scattering particles having differing sizes may be used. For example, a mixture of Aluminium oxide particles having a size of 0.3 μm and Aluminium oxide particles having a size of 3 μm may be used. Other sizes may be used according to the desired level of scattering. The scattering particles may form between 0% and 3.13% by weight of the TMM according to the desired ultrasound scattering level. The type of scattering particles included in this range may be chosen according to the desired properties. For example, they may be 0% to 1.06% for silicon carbide, or 0% to 3.13% for 3 μm aluminum oxide particles. In other embodiments, any other additional or alternative scattering particles known in the art may be used.

In some embodiments, the mixture may further comprise a preservative. The preservative may be included in the mixture to prevent bacterial invasion of the TMM. In some embodiments, the preservative may comprise Benzalkonium chloride (BC). In other embodiments, the preservative may comprise a mixture of sulphanic acid and oxalic acid. In other embodiments the preservative may be or comprise mould and mildew.

In some embodiments, the mixture may further comprise potassium chloride or sodium chloride. This may help stop the strands of the carrageen powder used to form the gelling agent from being hydrophobic and not mixing with the water.

An example of the different components and their relative quantities which can be included in the TMM forming mixture is summarised in the following table:

TABLE 1
Summary of TMM forming mixture components

| Materials | Percentage by weight of TMM (%) |
|---|---|
| Konjac | 1.05 to 1.95 |
| Carrageen | 1.05 to 1.95 |
| Si C | 0 to 1.06 |
| Al₂O₃ (0.3 μm) | 0 to 2.98 |
| Al₂O₃ (3 μm) | 0 to 3.13 |
| BC | 0.29 to 0.46 |
| Potassium chloride | 0.45 to 0.7 |
| Water | 61 to 94 |
| Glycerol | 0 to 11 |
| Oil and surfactant (fat part) | 0 to 35 |
| Silicone emulsion | 0 to 10 |

The quantities of the components included in TMM forming mixture may be varied independently of each other (e.g. one or more of the components may be restricted to one of the ranges or values above, whereas the quantities of the one or more other components may be unrestricted). In some embodiments, the TMM may include further components in addition to those listed in Table 1. In yet other embodiments, the TMM may consist essentially of only the components listed in Table 1 (e.g. components that do not materially affect the visual appearance of an ultrasound image or the haptic properties of the TMM, e,g. a preservative, may also be present). In yet other embodiments, the TMM consists of only the components listed in Table 1 (e.g. no other components are present).

The percentages and ratios given herein are by weight. The quantities (e.g. percentage weight) and relative ratios of components given herein relate to the final TMM rather than relative quantities of components added to the mixture from which the TMM is formed.

### Tissue Mimicking Material

In another aspect, the present disclosure provides a tissue mimicking material (TMM). The TMM of this aspect may be used as both an imaging phantom and a biopsy training device. The TMM may be used to simulate any desired human or animal tissue or other anatomical structures.

The TMM is formed using Konjac and Carrageen acting as a gelling agent along with water acting as a solvent as is known in the art. The TMM of the present invention may be formed from a number of portions having different properties to each other to simulate complex anatomical structures. The portions may be formed by different layers of the TMM (mimicking skin or fat layers, for example) or separate distinct smaller areas (mimicking lesions, for example). The TMM of the present invention generally comprises: a first portion formed from a mixture comprising: Konjac, Carrageen, and water; and a second portion also formed from a mixture comprising Konjac, Carrageen, and water. The ratio of Konjac to Carrageen in the first portion is different from the ratio of Konjac to Carrageen in the second portion. By having different ratios of Konjac and Carrageen the haptic feel of different parts of the TMM may be different from each other. This may provide a more realistic representation of human or animal tissue.

In some embodiments, the ratio of the weight of the Konjac to Carrageen in the first and/or the second portion is in a range of 10:90 to 90:10. This has been found to provide a realistic level of haptic feedback for a number of different types of human (or animal) tissue.

In some embodiments, the ratio of the weight of the Konjac to Carrageen in the first portion is chosen from any one of: 40:60, 50:50, 60:40 and 65:35, and the ratio of the weight of the Konjac to Carrageen in the second portion is chosen from another different one of: 40:60, 50:50, 60:40 and 65:35. The ratios may be chosen to provide a realistic simulation of the desired type of tissue. As discussed in more detail as follows, these ratios of Konjac to Carrageen may be used to mimic glandular tissue, fat layers, muscle tissue, ligament tissue, malignant lesions, benign lesions, fibro adenoma, anechoic lesions or hyperechoic lesions.

As discussed above, the proportion of gelling agent used may be chosen in order to provide a resulting TMM having the desired consistency and haptic properties. The total amount of Konjac and Carrageen in the first portion and/or the total amount of Konjac and Carrageen in the second portion may be in the range of 1.5% to 5% by weight of the respective first or second portions. This may provide a realistic level of haptic feedback.

As discussed above, the TMM may further comprise silicone emulsion to provide improved appearance in ultrasound images. The amount of silicone emulsion may vary between different portions of the TMM according to the type of tissue being replicated. As discussed above, each of the portions of the TMM may comprise silicon emulsion forming 3% to 10% of the total weight of the respective portion of the TMM. Specific values within this range may be chosen to simulate certain tissue types. In some embodiments, the silicon emulsion may form between 3.5% and 4.3% by weight, and preferably 3.92% by weight, of one of the portions of the TMM in order to mimic glandular tissue. In some embodiments, the silicon emulsion may form between 6.7% and 10% by weight, and preferably 8.37% by weight, of one of the portions of the TMM so as to mimic malignant lesions.

The materials and their relative quantities forming each of the TMM portions may be tailored according to the type of tissue or anatomical structure that is to be simulated by the TMM. The TMM may be used to mimic a wide variety of different types of tissue and anatomical structures as required by a suitable choice of materials or properties. In some embodiments, additional components or structures may be added to the TMM to provide further anatomical realism. For example, solid materials may be added to represent bone.

The TMM may be formed by any one or more of the following portions: a) at least one fat layer mimicking portion; b) at least one glandular tissue mimicking portion; c) at least one Cooper Ligament mimicking portion; d) at least one pectoral muscle mimicking portion e) at least one malignant lesion mimicking portion f) at least one benign lesion mimicking portion; g) at least one anechoic lesion mimicking portion; and h) at least one hyperechoic lesion mimicking portion. By combining various different portions from those listed (or others) different anatomies may be mimicked. The TMM may, for example, be used to mimic breast tissue. In other embodiments, the TMM may be used to mimic other tissue such as liver tissue, prostate tissue, abdominal tissue, kidney tissue, thyroid tissue or uterus tissue.

In one embodiment, the TMM may be specifically tailored for the simulation of breast tissue. In order to provide an anatomically accurate representation of the breast, the TMM may be formed from:
a fat layer mimicking portion;
a glandular tissue mimicking portion;
a Cooper Ligament mimicking portion;
a pectoral muscle mimicking portion; and
and any one or more of:
at least one malignant lesion mimicking portion;
at least one benign lesion mimicking portion;
at least one anechoic lesion mimicking portion; and
at least one hyperechoic lesion mimicking portion.

This choice of portions forming the TMM replicates the real shape of the breast complete with relevant internal breast tissue structures. It further includes a range of pathologies typically encountered in breast cancer patients. The size and shape of each of the portions is adapted according to the typical size and shape of the tissue types being mimicked. The materials used for each of the portions are chosen to exhibit properties from both an ultrasound and haptic feedback point of view, which replicates the real situation in vivo. The stiffness of lesion mimicking portions, for example, gives much information about their type. The stiffer a lesion is, the higher is the probability for it to be malignant. It is therefore important to provide realistic simulation of haptic feedback to provide accurate training.

A skin mimicking portion or layer may also be provided to give further anatomical realism. Such a skin layer may be formed, for example, from silicon and glycerol.

Further details of portions a) to h) from which the TMM may be formed are provided as follows.

### a) Fat Layer Mimicking Portion:

The fat layer mimicking portion may comprise Konjac, Carrageen and water, and may further comprise one or more scattering particles, oil and a surfactant. The surfactant may act to allow the fat and water to be mixed uniformly together, and in some embodiments may not be required. For the fat layer mimicking portion, the weight of the Konjac and Carrageen is in a ratio of 40:60.

The relative amount of oil included in the fat mimicking layer may be chosen according to the tissue being mimicked and may vary from 10 to 35% by weight of the fat mimicking portion. In one embodiment, the fat layer mimicking portion may comprise 35% by weight of oil. The percentage of the oil to the other constituents of the fat mimicking layer may be varied to get the speed of sound as close as possible to the desired value corresponding to that of the tissue being mimicked. A value of 35% may be chosen to provide a speed of sound of 1480 m/s which is suitable for mimicking the breast fat layer. In other embodiments, other proportions of oil may be chosen to mimic other fat layer types.

In one embodiment, the fat layer mimicking portion may be adapted to mimic a fat layer found in the breast. In this embodiment, the fat mimicking portion may comprise:

TABLE 2a
Summary of the fat mimicking portion components

| | Percentage by | | Tolerance range |
|---|---|---|---|
| | weight of total | Effective mass | (% by weight of |
| | fat mimicking | in 1000 ml of | total fat |
| | portion | fat mimicking | mimicking |
| Component | (%) | portion | portion) |
| Fat component | 35 | 350 | 25-40 |
| TMM component | 65 | 650 | 60-75 |

TABLE 2b
Summary of the fat component materials

| | Percentage by | Effective | Tolerance range |
|---|---|---|---|
| Fat component | weight of fat | mass (g) | (% by weight of |
| material | component (%) | in 350 ml | fat component) |
| Oil | 80 | 280 | 75-90 |
| Surfactant | 20 | 70 | 10-25 |

TABLE 2c
Summary of the TMM component materials

| | Percentage by | Effective | Tolerance range |
|---|---|---|---|
| TMM component | weight of TMM | mass (g) | (% by weight of |
| material | component (%) | in 650 ml | TMM component) |
| Konjac | 1.2 | 7.8 | 0.9-1.35 |
| Carrageen | 1.8 | 11.7 | 1.65-2.1 |
| Si C | 0.53 | 3.445 | 0.477-1.06 |
| Al₂O₃ (0.3 μm) | 0.89 | 5.785 | 0.445-0.98 |
| Al₂O₃ (3 μm) | 0.96 | 6.24 | 0.48-1.06 |
| BC | 0.46 | 2.99 | — |
| Potassium chloride | 0.7 | 4.55 | — |
| Water | 93 | 604 | 90.9-95.07 |

Table 2a shows the components used to form the fat mimicking portion—a fat component and a TMM component. FIG. 2b provides a breakdown of the materials making up the fat component of the fat mimicking portion. Table 2c gives a breakdown of the materials making up the TMM component of the fat mimicking portion. The quantities of materials in the TMM part have been scaled down to allow for the addition of the oil and surfactant. In the described embodiment, the weight of the oil and surfactant may be in the ratio of 80:20. Any suitable type of oil and surfactant may be used. The oil may be olive oil, for example. The surfactant may be Synperonic A7. In other embodiments, any other suitable surfactant may be used as would be apparent to the skilled person, or in some embodiment it may not be required.

Tests by the inventors on three batches of the fat layer mimicking portion formed using the components and quantities above produced the following results:

TABLE 3
Test results of the fat mimicking portion

| | Velocity | Velocity | Attenuation | Attenuation |
|---|---|---|---|---|
| Batch | (m/s) | error | (dB/cm/MHz) | error |
| B1 | 1487 | 0.13 | 0.75 | 0.014 |
| B2 | 1484 | 0.27 | 0.82 | 0.013 |
| B3 | 1485 | 0.41 | 0.77 | 0.011 |
| Overall | 1485 | 0.27 | 0.78 | 0.012 |

Alternatively to the embodiment above where the weight of the Konjac and Carrageen is in a ratio of 40:60, the ratio may instead be 50:50. In this embodiment, the fat mimicking portion may comprise:

TABLE 2d
Summary of the TMM component materials

| | Percentage by |
|---|---|
| TMM component | weight of TMM |
| material | component (%) |
| Konjac | 1.5 |
| Carrageen | 1.5 |
| Si C | 0.53 |
| Al₂O₃ (0.3 μm) | 0.89 |
| Al₂O₃ (3 μm) | 0.96 |
| Glycerol | 11 |
| Mould and mildew | 2 |
| Sodium chloride | 0.7 |

Table 2d gives a breakdown of the materials making up the TMM component of the fat mimicking portion, as an alternative to the breakdown given in Table 2c.

(For these measures, and others given herein, the speed of sound in water was 1486.588 at 21.6 degrees.)

### b) Glandular Tissue Mimicking Portion:

The glandular tissue mimicking portion may comprise Konjac, Carrageen and water, and may further comprise one or more scattering particles, glycerol and silicone emulsion. For the glandular tissue mimicking portion, the weight of the Konjac and Carrageen is in a ratio of 60:40.

The glandular tissue mimicking portion may be composed of water and silicone emulsion in a ratio of: about 95% of water and about 5% silicone emulsion (% by weight). The silicone emulsion and water together may both make 84% by weight of the whole TMM and the other materials may remain in the proportion used for the fat layer mimicking portion (e.g. some of water is removed to include the silicone emulsion). The silicone emulsion may be added to fulfill the attenuation requirements of the glandular tissue which is about 2 dB/cm/MHz, as described above. In one embodiment, the amount of silicone emulsion may be about 3.9% by weight of the glandular tissue mimicking portion.

In one embodiment, the glandular tissue mimicking portion may be adapted to mimic a glandular tissue layer found in the breast. In this embodiment, the glandular tissue mimicking portion may comprise:

TABLE 4
Summary of the glandular tissue mimicking portion components

| | Percentage by | | |
|---|---|---|---|
| | weight of | | Tolerance range |
| | glandular tissue | | (% by weight of |
| | mimicking | Effective | glandular tissue |
| | portion | mass | mimicking |
| Materials | (%) | (g) | portion) |
| Konjac | 1.68 | 16.8 | 1.51-1.85 |
| Carrageen | 1.12 | 11.2 | 1-1.23 |
| Si C | 0.49 | 4.9 | 0.44-0.54 |
| Al₂O₃ (0.3 μm) | 2.98 | 29.8 | 2.68-3.28 |
| Al₂O₃ (3 μm) | 3.13 | 31.3 | 2.8-3.44 |
| Glycerol | 11.19 | 111.9 | — |
| BC | 0.43 | 43 | — |
| Potassium chloride | 0.65 | 6.5 | — |
| Water | 74.41 | 744.1 | 74-75 |
| Silicone emulsion | 3.92 | 39.2 | 3.5-4.3 |

Tests by the inventors on three batches of the glandular tissue mimicking portion produced using the components above produced the following results:

TABLE 5
Test results of the glandular tissue mimicking portion

| | Velocity | Velocity | Attenuation | Attenuation |
|---|---|---|---|---|
| Batch | (m/s) | error | (dB/cm/MHz) | error |
| B1 | 1527 | 0.55 | 1.79 | 0.019 |
| B2 | 1534 | 0.69 | 1.90 | 0.063 |
| B3 | 1534 | 0.89 | 1.85 | 0.031 |
| Overall | 1532 | 0.71 | 1.87 | 0.038 |

### c) Cooper Ligament Mimicking Portion:

The Cooper Ligament mimicking portion may comprise Konjac, Carrageen and water, and may further comprise one or more scattering particles and glycerol. For this portion the weight of the Konjac and Carrageen may be in a ratio of 50:50. In some embodiments, the Cooper Ligament mimicking portion could be used to mimic other ligaments found in other parts of the body.

In one embodiment, the Cooper Ligament mimicking portion may be adapted to mimic the Cooper Ligament found in the breast. The Cooper ligament may be mimicked by an hyperechoic TMM that can be positioned between a fat mimicking portion and a glandular tissue mimicking portion. In this embodiment, the Cooper Ligament mimicking portion may have a composition as follows:

TABLE 6
Summary of the Cooper Ligament mimicking portion components

| | Percentage by | | |
|---|---|---|---|
| | weight of | | Tolerance range |
| | Cooper Ligament | | (% by weight of |
| | mimicking | Effective | Copper Ligament |
| | portion | mass | mimicking |
| Materials | (%) | (g) | portion) |
| Konjac | 1.44 | 14.43 | 1.3-1.58 |
| Carrageen | 1.44 | 14.43 | 1.3-1.58 |
| Si C | 1.02 | 10.20 | 0.92-1.12 |
| Al₂O₃ (0.3 μm) | 1.71 | 17.13 | 1.54-1.88 |
| Al₂O₃ (3 μm) | 1.85 | 18.48 | 1.66-2.04 |
| Glycerol | 10.59 | 105.85 | — |
| BC | 0.44 | 4.43 | — |
| Potassium chloride | 0.67 | 6.74 | — |
| Water | 80.83 | 808.31 | 80-81.6 |

Tests by the inventors on the Cooper Ligament mimicking portion produced using the components above produced the following results:

TABLE 7
Test results of the Cooper Ligament mimicking portion

| | Velocity | Velocity | Attenuation | Attenuation |
|---|---|---|---|---|
| Batch | (m/s) | error | (dB/cm/MHz) | error |
| Overall | 1565 | 1 | 1.9 | 0.05 |

### d) Pectoral Muscle Mimicking Portion:

The pectoral muscle mimicking portion may comprise Konjac, Carrageen and water, and may further comprise one or more scattering particles and glycerol. For the pectoral muscle mimicking portion the weight of the Konjac and Carrageen may be in a ratio of 50:50. In some embodiments, the pectoral muscle mimicking portion could be used to mimic other types of muscle tissue found in other parts of the body.

In one embodiment, the pectoral muscle mimicking portion may be adapted to mimic the pectoral muscle tissue layer found in the breast. In this embodiment, the pectoral muscle mimicking portion may comprise:

TABLE 8
Summary of the pectoral muscle mimicking portion components

| | Percentage by | | |
|---|---|---|---|
| | weight of | | Tolerance range |
| | pectoral muscle | | (% by weight of |
| | mimicking | Effective | Pectoral muscle |
| | portion | mass | mimicking |
| Materials | (%) | (g) | portion) |
| Konjac | 1.48 | 14.77 | 1.33-1.63 |
| Carrageen | 1.48 | 14.77 | 1.33-1.63 |
| Si C | 0.52 | 5.22 | 0.47-0.57 |
| Al₂O₃ (0.3 μm) | 0.88 | 8.77 | 0.79-0.97 |
| Al₂O₃ (3 μm) | 0.95 | 9.45 | 0.86-1.05 |
| Glycerol | 10.83 | 108.33 | — |
| BC | 0.45 | 4.53 | — |
| Potassium chloride | 0.69 | 6.89 | — |
| Water | 82.73 | 827.26 | 82.2-83.3 |

Tests by the inventors on three batches of the pectoral muscle mimicking portion produced using the components above produced the following results:

TABLE 9
Test results of the pectoral muscle mimicking portion

| | Velocity | Velocity | Attenuation | Attenuation |
|---|---|---|---|---|
| Batch | (m/s) | error | (dB/cm/MHz) | error |
| B1 | 1548 | 1.85 | 0.54 | 0.04 |
| B2 | 1552 | 0.90 | 0.55 | 0.03 |
| B3 | 1545 | 0.40 | 0.49 | 0.01 |
| Overall | 1548 | 1.05 | 0.53 | 0.03 |

### e) Malignant Lesion Mimicking Portion:

The malignant lesion mimicking portion may comprise Konjac, Carrageen and water and may further comprise one or more scattering particles, glycerol and silicone emulsion. For the malignant lesion mimicking portion the weight of the Konjac and Carrageen is in a ratio of 40:60. The malignant lesion mimicking portion may also be used to form a fibro-adenoma mimicking portion.

The malignant lesion mimicking portion may contain less scatterers than the other portions forming the TMM so may appear darker. The backscatter value of these lesions is −3 dB. To satisfy the attenuation value found in literature for real malignant lesions, the malignant lesion mimicking portion contains about 8% by weight of silicone emulsion.

In one embodiment, the malignant lesion mimicking portion may be adapted to mimic the malignant lesions found in the breast. In this embodiment, the malignant lesion mimicking portion may comprise:

TABLE 10
Summary of the malignant lesion mimicking portion components

| | Percentage by | | |
|---|---|---|---|
| | weight of | | Tolerance range |
| | malignant lesion | | (% by weight of |
| | mimicking | Effective | malignant lesior |
| | portion | mass | mimicking |
| Materials | (%) | (g) | portion) |
| Konjac | 1.20 | 11.96 | 1.08-1.32 |
| Carrageen | 1.79 | 17.94 | 1.61-1.97 |
| SiC | 0.26 | 2.64 | 0.23-0.29 |
| Al₂O₃ (0.3 μm) | 0.44 | 4.43 | 0.4-0.48 |
| Al₂O₃ (3 μm) | 0.48 | 4.78 | 0.43-0.53 |
| Water | 75.34 | 753.36 | 75-76 |
| Silicone emulsion | 8.37 | 83.71 | 6.7-10 |
| Glycerol | 10.96 | 109.62 | — |
| BC | 0.46 | 4.58 | — |
| Potassium chloride | 0.70 | 6.98 | — |

Tests by the inventors on the malignant lesion mimicking portion produced using the components above produced the following results:

TABLE 11
Test results of the malignant lesion mimicking portion

| | Velocity | Velocity | Attenuation | Attenuation |
|---|---|---|---|---|
| Batch | (m/s) | error | (dB/cm/MHz) | error |
| Overall | 1492 | 1 | 0.99 | 0.05 |

### f) Benign Lesion Mimicking Portion:

The Benign lesion mimicking portion may comprise Konjac, Carrageen and water and may further comprise one or more scattering particles and glycerol, wherein the weight of the Konjac and Carrageen is in a ratio of 65:35.

The benign lesion mimicking portion may also contain less scatterers than the other portions of the TMM and may be softer than the malignant lesions. The backscatter value of the benign lesion mimicking portion may be −2 dB.

In one embodiment, the benign lesion mimicking portion may be adapted to mimic the benign lesions found in the breast. In this embodiment, the benign lesion mimicking portion may comprise:

TABLE 12
Summary of the benign lesion mimicking portion components

| | Percentage by | | |
|---|---|---|---|
| | weight in | | Tolerance range |
| | benign lesion | | (% by weight of |
| | mimicking | Effective | benign lesion |
| | portion | mass | mimicking |
| Materials | (%) | (g) | portion) |
| Konjac | 1.94 | 19.38 | 1.75-2.13 |
| Carrageen | 1.04 | 10.44 | 0.94-1.14 |
| Si C | 0.32 | 3.16 | 0.29-0.35 |
| Al₂O₃ (0.31 μm) | 0.53 | 5.31 | 0.48-0.58 |
| Al₂O₃ (3 μm) | 0.57 | 5.73 | 0.51-0.63 |
| Water | 83.50 | 834.99 | 83-84 |
| Glycerol | 10.93 | 109.34 | — |
| BC | 0.46 | 4.57 | — |
| Potassium chloride | 0.70 | 6.96 | — |

Tests by the inventors on three batches of the benign lesion mimicking portion produced using the components above produced the following results:

TABLE 13
Test results of benign lesion mimicking portion

| | Velocity | Velocity | Attenuation | Attenuation |
|---|---|---|---|---|
| Batch | (m/s) | error | (dB/cm/MHz) | error |
| B1 | 1557 | 0.43 | 0.34 | 0.016 |
| B2 | 1568 | 0.86 | 0.31 | 0.016 |
| B3 | 1567 | 0.99 | 0.27 | 0.015 |
| Overall | 1564 | 0.76 | 0.31 | 0.016 |

### g) Anechoic Lesion Mimicking Portion:

The anechoic lesion mimicking portion may comprise Konjac, Carrageen, and water and may further comprise glycerol. The weight of the Konjac and Carrageen may be in a ratio of 40:60 for the anechoic lesion mimicking portion. The anechoic lesion mimicking portion does not require the scattering particles which are responsible for the backscattering.

In one embodiment, the anechoic lesion mimicking portion may be adapted to mimic the anechoic lesions found in the breast. In this embodiment, the anechoic lesion mimicking portion may comprise:

TABLE 14
Summary of the anechoic lesion mimicking portion components

| | Percentage by | | |
|---|---|---|---|
| | weight in | | Tolerance range |
| | anechoic lesion | | (% by weight of |
| | mimicking | Effective | anechoic lesion |
| | portion | mass | mimicking |
| Materials | (%) | (g) | portion) |
| Konjac | 1.2 | 12.33 | 1.1-1.35 |
| Carrageen | 1.8 | 18.49 | 1.67-2.0 |
| Water | 86.28 | 862.78 | 85.9-86.59 |
| Glycerol | 9.56 | 95.6 | — |
| BC | 0.46 | 4.72 | — |
| Potassium chloride | 0.7 | 7.19 | — |

Tests by the inventors on the anechoic lesion mimicking portion produced using the components above produced the following results:

TABLE 15
Test results of the anechoic lesion mimicking portion

| | Velocity | Velocity | Attenuation | Attenuation |
|---|---|---|---|---|
| Batch | (m/s) | error | (dB/cm/MHz) | error |
| Overall | 1545 | 1 | 0.15 | 0.05 |

### h) Hyperechoic Lesion Mimicking Portion:

The hyperechoic lesion mimicking portion may comprise Konjac, Carrageen and water and may further comprise one or more scattering particles and glycerol. The weight of the Konjac and Carrageen may be in a ratio of 40:60 for the Hyperechoic lesion mimicking portion. The materials and proportions of materials may be chosen such that the hyperechoic lesion mimicking portion is +3 dB more echoic than the rest of the TMM.

In one embodiment, the hyperechoic lesion mimicking portion may be adapted to mimic the hyperechoic lesions found in the breast. In this embodiment, the hyperechoic lesion mimicking portion may comprise:

TABLE 16
Summary of the hyperechoic lesion mimicking portion components

| | Percentage by | | |
|---|---|---|---|
| | weight in | | Tolerance range |
| | hyperechoic lesion | | (% by weight of |
| | mimicking | Effective | hyperechoic lesion |
| | portion | mass | mimicking |
| Materials | (%) | (g) | portion) |
| Konjac | 1.15 | 11.55 | 1.04-1.27 |
| Carrageen | 1.73 | 17.32 | 1.56-1.9 |
| Si C | 1.02 | 10.20 | 0.92-1.12 |
| Al₂O₃ (0.3 μm) | 1.71 | 17.13 | 1.54-1.88 |
| Al₂O₃ (3 μm) | 1.85 | 18.48 | 1.67-2.0 |
| Water | 80.83 | 808.31 | 80-81.56 |
| Glycerol | 10.59 | 105.85 | — |
| BC | 0.44 | 4.43 | — |
| Potassium | 0.67 | 6.74 | — |
| chloride | | | |

Tests by the inventors on the hyperechoic mimicking portion produced using the components above produced the following results:

TABLE 17
Test results of hyperechoic mimicking portion

| | Velocity | Velocity | Attenuation | Attenuation |
|---|---|---|---|---|
| Batch | (m/s) | error | (dB/cm/MHz) | error |
| Overall | 1594 | 1 | 0.68 | 0.05 |

A summary of the different TMM portions described above after being tested to determine the material speed of sound, attenuation and elasticity are provided in the table below. The fibro-adenoma layer may be formed from the same constituent parts and relative quantities as the malignant lesions described above.

TABLE 18
Summary of test results for various TMM portions

| | | | Attenuation coefficient | | | |
|---|---|---|---|---|---|---|
| | | | dB · cm⁻¹ · MHz⁻¹ | | Elasticity | |
| | Speed of sound m/s | | since 1 value is at 5 MHz | | Young's Modulus, E | |
| Tissue type | Ideal | TMM | Ideal | TMM | Ideal | TMM |
| Glandular tissue | 1553 ± 35 | 1532 ± 0.7 | 2.0 ± 0.8 | 1.87 ± 0.04 | 35 ± 14 | 50 ± 1.3 |
| (Parenchymal, adipose | | | @7 MHz | | | |
| and fibrous tissues) | | | | | | |
| TMM portion (b) | | | | | | |
| Subcutaneous Fat | 1479 ± 32 | 1486 ± 0.3 | 0.6 ± 0.1 | 0.76 ± 0.01 | 22 ± 12 | 43 ± 2 (ii) |
| TMM portion (a) | | | @7 MHz | | | |
| Pectoral Muscle | 1545 ± 5 | 1548 ± 1.05 | | 0.53 ± 0.03 | | 64.7 ± 1.7 (iii) |
| TMM portion (c) | | | | | | |
| Areola | — | — | 1.1 @ 5 MHz | | | |
| Cooper Ligaments | — | — | — | | | |
| TMM portion (d) | | | | | | |
| Malignant Lesions | 1550 ± 35 | 1492 ± 0.41 | 1.0 ± 0.3 | 0.99 ± 0.03 | 68-123 | 103.4 ± 1.6 |
| TMM portion (e) | | | @7 MHz | | | |
| Fibro-adenoma | 1584 ± 27 | 1492 ± 0.41 | — | 0.99 ± 0.03 | 100.5 ± 39.6 | 103.4 ± 1.6 |
| Benign lesions | — | 1564 ± 0.76 | | 0.33 ± 0.016 | 25-50 | 42.80 ± l |
| TMM portion (f) | | | | | | |

The embodiments of the TMM portions a) to h) described above are to be understood as preferred examples only. Each of the components described in Tables 2a-c, 4, 6, 8, 10, 12, 14 and 16 above may be varied in amount independently of each other, or may be omitted entirely. In some embodiments, additional components may be present in any of the TMM portions described above and in Tables 2a-c, 4, 6, 8, 10, 12, 14 and 16. In yet other embodiments, the fat layer mimicking portion; glandular tissue mimicking portion; Cooper Ligament mimicking portion; pectoral muscle mimicking portion; malignant lesion mimicking; portion benign lesion mimicking portion; anechoic lesion mimicking portion; and hyperechoic lesion mimicking portion may consist only of the components listed in tables 2a-c, 4, 6, 8, 10, 12, 14 and 16 above, respectively. In other embodiments, they may consist essentially of the components listed in Tables 2a-c, 4, 6, 8, 10, 12, 14 and 16 i.e. further components may be present that do not materially affect the characteristics (e.g. the ultrasound imaging or haptic properties) of the TMM.

The percentages and ratios given above are by weight. The quantities (e.g. percentage weight) and relative ratios of components given herein relate to the final TMM rather than relative quantities of components added to the mixture from which the TMM is formed.

### Refillable Biopsy Training Device

In another aspect, the present application discloses a biopsy training device, an embodiment of which is shown in FIG. 1a. In this embodiment, the biopsy training device (100) comprises: a skin mimicking layer 102; and a fibroglandular tissue mimicking portion 104 comprising one or more lesion mimicking portions. The fibroglandular mimicking portion 104 and the skin mimicking layer 102 are visible in the cut away view shown in FIG. 1b. These components are also shown separately in FIGS. 1c and 1d. The fibroglandular tissue mimicking portion 104 is removably coupled to the skin mimicking layer 102. In some embodiments, any other additional tissue mimicking portions can be provided in the biopsy training device, including any one or more of the TMM portions described in the previous section.

In the embodiment shown in FIGS. 1a and 1b, a two part training device is formed by the outer skin layer and an inner “disposable” fibroglandular TMM portion containing the lesions. During use the fibroglandular portion will undergo destruction due to the biopsy practice at a faster rate than the skin mimicking layer. The use of a removable fibroglandular mimicking portion means that after a number of uses the fibroglandular mimicking portion can be removed and replaced. This can be done once the lesion mimicking portions have been removed or damaged to such an extent that the device would otherwise have to be disposed of entirely. A “refillable” training device can therefore be provided having a longer lifespan of use compared to those of the prior art.

In the described embodiment, the biopsy training device 100 comprises a backing member 106 arranged to couple to the skin mimicking layer 102. A backing member according to the described embodiment is shown in FIG. 1e. The backing member 106 may be formed from a backing plate arranged to couple to the skin mimicking layer 102 by any suitable means known in the art. In some embodiments, the backing member may comprise a grid structure as shown in FIG. 1b to replicate rib bones. In other embodiments, the backing member 106 may have any other suitable shape and may, for example, be shaped to correspond to the shape of the skin mimicking layer 102.

The skin mimicking layer 102 and the backing member 106 may form a housing when coupled together in which the fibroglandular tissue mimicking portion 104 is received. The fibroglandular tissue mimicking portion 104 may be coupled to an inside wall of the skin mimicking layer 102 by a coupling liquid comprising water, glycerol and Benzalkonium chloride (BC).

The fibroglandular TMM portion may be removed from the biopsy training device 100 by removing the backing member 106 from the device and pushing the fibroglandular mimicking portion 104 out of the skin mimicking layer 102. A new replacement fibroglandular portion can then be inserted into the space left behind and optionally coupled to the skin mimicking layer 102 using ultrasound coupling gel. The backing member 106 can then be recoupled to the skin mimicking layer 102 to hold the fibroglandular tissue mimicking portion 104 in place. The biopsy training device is then ready for use with a new undamaged set of lesions.

In some embodiments, the lesion mimicking portions may comprise a colour dye to provide a visual indication of when the sample of tissue has been successfully removed. The colour dye may be used to indicate if the correct type of lesion was biopsied. For example, a different colour dye may be used indicate a correct biopsy compared to the colour used to indicate an incorrect biopsy. In one embodiment, green dye may be used to indicate correct biopsy and a red dye may be used to indicate incorrect biopsy.

### Method of Manufacturing a Tissue Mimicking Material

In another aspect, the present application also discloses a method of manufacturing a TMM. A method 200 of manufacturing a tissue mimicking material according to one embodiment is shown in FIG. 2. In this embodiment, the method generally comprises the following steps: a) adding 202 one or more scattering particles to water; b) adding 204 gelling agent to the mixture formed in step a); c) heating 206 the mixture formed in part b); d) forming 208 the mixture formed in part c) into a desired shape. In step b) the mixture is mixed at a first rate during the addition of the gelling agent. In step c) the mixture is mixed at a second mixing rate while being heated. The second mixing rate is slower than the first. In some embodiments, the step of adding the scattering particles may be omitted if they are not required in the resulting TMM.

By using a faster mixing rate while adding the gelling agent compared to during heating helps avoid clumping of the gelling agent while it is being added, and also allows a homogeneous solution without air bubbles to be formed by the slower mixing rate during the heating stage.

In one embodiment, the first mixing rate may be between 200 and 260 rpm and the second mixing rate may be between 110 and 160 rpm. These ranges of mixing rates have been found to provide good mixing of the gelling agent and a homogeneous resulting TMM. By “mixing rate” we mean, for example, the rate of rotation of a mixing device (e.g. a stirrer or the like) used to mix the mixture.

The water used to produce the TMM may be deionised water. In some embodiments, the water is pre-weighed to ensure the correct quantity is used as would be apparent to the skilled person.

In some embodiments, in step a), the addition 202 of one or more scattering particles to the water may comprise adding a pre-weighed quantity of scattering particles to the water. The scattering particles may comprise one or more of silicon carbide particles or aluminium oxide particles or any mixture thereof as described above. The scattering particles may be sieved before being added to the water to avoid clumping during the manufacturing process. This may be done using a kitchen sieve or the like if production is on a small scale, or any other appropriate sieve for larger scale production. The scattering particles may be added by slowly pouring them into the water using any suitable means apparent to the skilled person.

In some embodiments, before the addition of the scattering particles to the water, the method 100 may further comprise adding potassium chloride to the water. In such an embodiment, a pre-weighed quantity of potassium chloride may be added to the water and the resulting mixture mixed such that the potassium chloride dissolves and is distributed within the liquid. In other embodiments, the potassium chloride may not be required and this step may be omitted.

Once the scattering particles have been added, the TMM forming mixture is mixed to homogeneously mix the powders and water (and potassium chloride solution where used). During this mixing, the mixing speed may be about 220 rpm. This speed has been found to be high enough to maintain the scattering particles in suspension but slow enough to avoid the production of air bubbles in the mixture.

In some embodiments, the method 200 may further comprise adding silicone emulsion to the water in order to produce a silicone emulsion containing TMM as described above. The silicone emulsion may be added before the addition of the scattering particles. In other embodiments, the silicone emulsion may be added at any other suitable point in the method. In other embodiments, the silicone emulsion may not be required and this step may be omitted.

In one embodiment, the gelling agent added 204 to the water in step b) may be in the form of a dry powder. The gelling agent may comprise a mixture of Konjac and Carrageen powders as required to form the desired TMM (as described in the previous sections). In other embodiments, any other suitable gelling agent may be used. The quantities of Konjac and Carrageen may be pre-weighed before being added to the water. In some embodiments, the gelling agent may be added to the water via a sprinkler loaded with the desired quantity of gelling agent. If using Konjac and Carrageen powders, the method may comprise mixing the gelling agent components before addition to the water. In this embodiment, no sieving is however required.

In some embodiments, the gelling agent may be added to the TMM forming mixture using a sprinkler arranged to disperse the gelling agent powders. The gelling agent may be added over a period of time while the TMM forming mixture is being mixed at the first mixing rate. In some embodiments, the gelling agent powders may be added over a period of 2 to 4 minutes. In other embodiments, the gelling agent may be added at a rate appropriate for the quantity of TMM being made. Furthermore, the gelling agent may be added using any means suitable for the type of gelling agent being used.

The first mixing rate may be in a range between about 200 and about 260 rpm. These rates have been found to help reduce clumping of the gelling agent once it has been added. This may be advantageous in embodiments where the gelling agent comprises a mixture of Carrageen and Konjac powders which may otherwise tend to form clumps. In one preferred embodiment, the first mixing rate may be 220 rpm to provide good mixing with reduced clumping.

While the gelling agent is being added, the TMM forming mixture may be at ambient temperature. Following the addition of the gelling agent, the resulting mixture is then heated 106 to a temperature of 95 to 100° C. The TMM forming mixture may be maintained at this temperature for a period of time while the gelation process takes place. This provides a matrix structure for the particles to be uniformly distributed through. In some embodiments, the mixture may be heated for a duration of about 1 hour.

The TMM forming mixture may be heated by placing a vessel in which it is contained in a water bath set to the appropriate temperature. In one embodiment, the temperature of the water bath may be 94° C. The temperature of the mixture may be monitored (using a thermocouple or the like) regularly (e.g. every 5 minutes) until the mixture reaches the desired temperature. The mixture may then be maintained at the desired temperature for around one hour. In other embodiments, any other suitable heating means may be used according to the volume of mixture being produced.

During heating 206, the TMM forming mixture is mixed at the second mixing rate. The second mixing rate may be chosen to provide homogeneous mixing of the metallic particles in the viscus TMM mixture without creating air bubbles. In one embodiment, the second mixing rate may be chosen in the range of 110 and 160 rpm. In a preferred embodiment, the second mixing rate may be 120 rpm±2 rpm. This has been found to provide homogenous mixing with no adverse effect on the composition of the final TMM. Speeds of less than 100 rpm have been found to result in an inhomogeneous TMM with an associated adverse effect on the acoustic properties.

In some embodiments, the method may further comprise adding glycerol to the heated mixture formed in step c). In some embodiments, the glycerol may be heated before it is added, the glycerol preferably being heated to a temperature in the range of 70 to 90° C. This may help avoid the molten TMM congealing. In other embodiments, the glycerol may not be required and so this step may be omitted.

In some embodiments, Benzalkonium chloride (or other preservative such as sulphanic acid and oxalic acid) may be added to the glycerol before it is added to the TMM mixture (and before it is heated). In other embodiments, the Benzalkonium chloride may not be required and so this step may be omitted.

Once the glycerol has been added, the TMM mixture may continue to be heated to allow the glycerol solution to be absorbed. The TMM mixture may be heated for a further period of about 15 minutes, which has been found to provide suitable absorption. In other embodiments, this heating time may be altered according the TMM being made. Once the glycerol has been absorbed, the TMM mixture may be allowed to cool before being formed into the desired shape. The TMM mixture may, for example, be allowed to cool to a temperature of 90° C. before being shaped. This may be done by pouring the TMM mixture into one or more moulds as is known in the art such that it may set into the desired shape.

Alternatively, in embodiments where the preservative is not added to the glycerol before it is added to the TMM mixture (and before it is heated), once the glycerol solution has been added, the TMM mixture may continue to be heated to allow the glycerol solution to be absorbed. The TMM mixture may be heated for a further period of about 12 minutes, which has been found to provide suitable absorption. Mould and mildew may then be heated for about 2 minutes and added to the TMM and allowed to mix for about 2 minutes. In other embodiments, this heating time may be altered according the TMM being made. Once the glycerol has been absorbed, the TMM mixture may be removed from the water bath. The TMM may optionally be allowed to cool before being formed into the desired shape. The TMM mixture may, for example, be allowed to cool to a temperature of 90° C. before being shaped. This may be done by pouring the TMM mixture into one or more moulds as is known in the art such that it may set into the desired shape.

In some embodiments, the method may further comprise adding oil to the resulting mixture formed in step c). In some embodiments, the oil may be combined with a surfactant. In this embodiment, the method may be suitable to form an oil containing TMM such as the fat layer mimicking TMM portion described above. In other embodiments, these steps may be omitted if no oil content is required. Before being added, an appropriate quantity of the oil and surfactant may be blended to mix them thoroughly to obtain a uniform mixture.

The oil and surfactant may be added once the gelling agent has been added and the mixture heated for the desired time period. Before being added, the oil and surfactant mixture may also be heated. The oil and surfactant may preferably be heated to a temperature of greater than about 80° C. The oil and surfactant may be heated by placing it in the water bath used to heat the TMM forming mixture, or any other suitable heating means. During heating the oil and surfactant may be mixed at periodic intervals.

Once the oil and surfactant has been added, the resulting mixture may be mixed at a third mixing rate. The third mixing rate may be between 150 rpm and 200 rpm. In one embodiment, mixing at the third mixing rate comprises a first mixing period of 200 rpm and a second mixing period of 150 rpm. The duration of the first and second mixing periods may be chosen to provide adequate mixing. In one preferred embodiment, the first mixing period may be 5 minutes in duration, and the second mixing period may be 10 minutes in duration.

During addition of the oil and surfactant and/or during mixing at the third mixing rate the TMM mixture may continue to be heated (e.g. at about 90 degrees).

In some embodiments, the method may further comprise mixing the TMM forming mixture at a fourth mixing rate after mixing at the third mixing rate. While mixing at the fourth mixing rate the mixture may be allowed to cool (e.g. the heat source may be removed).

The fourth mixing rate may be in the range of 250 rpm to 320 rpm, and preferably may be about 290 rpm. This may help make the TMM more homogenous. The mixture may be mixed at the fourth mixing rate for a duration suitable to provide a homogeneous TMM. In some embodiments, this may be a duration of about 5 minutes. This time may be varied according to the volume and type of TMM being produced. After mixing at the fourth mixing rate, the TMM mixture may be formed into the desired shape as described above.

Various modifications will be apparent to the skilled person without departing form the scope of the claims. The features of any embodiment described in the sections above may be combined with any other embodiments described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination. The applicant hereby gives notice that new claims may be formulated to such features and/or combinations of such features during the prosecution of the present application or of any further application derived therefrom.

For the sake of completeness, it is also stated that the term “comprising” does not exclude other elements or steps, the term “a” or “an” does not exclude a plurality, a single processor or other unit may fulfil the functions of several means recited in the claims and any reference signs in the claims shall not be construed as limiting the scope of the claims.

1. A mixture for forming a tissue mimicking material, the mixture comprising:
a) a gelling agent;
b) water;
c) glycerol;
d) one or more scattering particles; and
e) silicone emulsion.

2. The mixture of claim 1, wherein the silicone emulsion comprises 3% to 10% of the total weight of the tissue mimicking material.

3. The mixture of claim 2, wherein:
the silicone emulsion comprises between 3.5% and 4.3% and preferably 3.92%, of the total weight of the tissue mimicking material and preferably the water comprises between 74% and 75%, and further preferably 74.41%, of the total weight of the tissue mimicking material, or
the silicone emulsion comprises between 6.7% and 10%, and preferably 8.37%, of the total weight of the tissue mimicking material and the water preferably comprises between 75% and 76%, and further preferably 75.34%, of the total weight of the tissue mimicking material.

4. The mixture claim 1, wherein the gelling agent comprises between 1.5% and 5% of the total weight of the tissue mimicking material, and preferably comprises 3% of the total weight of the tissue mimicking material.

5. The mixture of claim 1, wherein the gelling agent comprises a mixture of Konjac and Carrageen and, optionally or preferably, wherein the Carrageen is Carrageen-Kappa or Carrageen-Iota.

6. The mixture of claim 5, wherein the ratio of the weight of the Konjac to Carrageen in the tissue mimicking material is in the range of 10:90 to 90:10, and wherein the ratio is preferably any one of: 50:50, 60:40, 40:60 or 65:35.

7. The mixture of claim 1, wherein the one or more scattering particles are chosen from any one or more of:
i) Silicon carbide particles; or
ii) Aluminium Oxide particles.

8. The mixture of claim 1, further comprising a preservative, wherein the preservative is preferably Benzalkonium chloride, a mixture of sulphanic acid and oxalic acid, or mould and/or mildew.

9. The mixture of claim, further comprising potassium chloride and/or sodium chloride.

10. A tissue mimicking material comprising:
a first portion formed from a mixture comprising: Konjac, Carrageen, and water; and
a second portion formed from a mixture comprising Konjac, Carrageen, and water,
wherein the ratio of Konjac to Carrageen in the first portion is different from the ratio of Konjac to Carrageen in the second portion.

11. The tissue mimicking material of claim 10, wherein the ratio of the weight of the Konjac to Carrageen in the first and/or the second portion is in a range of 10:90 to 90:10.

12. The tissue mimicking material of claim 11, wherein:
the ratio of the weight of the Konjac to Carrageen in the first portion is chosen from any one of: 40:60, 50:50, 60:40 and 65:35, and
the ratio of the weight of the Konjac to Carrageen in the second portion is chosen from another one of: 40:60, 50:50, 60:40 and 65:35.

13. The tissue mimicking material of claim 10 wherein the total amount of Konjac and Carrageen in the first portion and/or the total amount of Konjac and Carrageen in the second portion is in the range of 1.5% to 5% by weight of the respective first or second portions.

14. The tissue mimicking material of claim 10, wherein the first portion and the second portion are chosen from any of the follow portions having different ratios of Konjac and Carrageen to each other:
a) a fat layer mimicking portion comprising the Konjac, Carrageen and water, and further comprising one or more scattering particles, oil and a surfactant, wherein the weight of the Konjac and Carrageen is in a ratio of 40:60;
b) a glandular tissue mimicking portion comprising the Konjac, Carrageen and water, and further comprising one or more scattering particles, glycerol and silicone emulsion, wherein the weight of the Konjac and Carrageen is in a ratio of 60:40;
c) a Cooper Ligament mimicking portion comprising the Konjac, Carrageen and water, and further comprising one or more scattering particles and glycerol, wherein the weight of the Konjac and Carrageen is in a ratio of 50:50;
d) a pectoral muscle mimicking portion comprising the Konjac, Carrageen and water, and further comprising one or more scattering particles and glycerol, wherein the weight of the Konjac and Carrageen is in a ratio of 50:50;
e) a malignant lesion mimicking portion comprising the Konjac, Carrageen and water and further comprising one or more scattering particles, glycerol and silicone emulsion, wherein the weight of the Konjac and Carrageen is in a ratio of 40:60;
f) a benign lesion mimicking portion comprising the Konjac, Carrageen and water and further comprising one or more scattering particles and glycerol, wherein the weight of the Konjac and Carrageen is in a ratio of 65:35;
g) an anechoic lesion mimicking portion comprising the Konjac, Carrageen, and water and further comprising glycerol, wherein the weight of the Konjac and Carrageen is in a ratio of 40:60; and
h) a hyperechoic lesion mimicking portion comprising the Konjac, Carrageen and water and further comprising one or more scattering particles and glycerol, wherein the weight of the Konjac and Carrageen is in a ratio of 40:60.

15. The tissue mimicking material of claim 14, wherein:
the silicone emulsion forms between 3.5% and 4.3% by weight and preferably 3.92% by weight of the glandular tissue mimicking portion, and/or
the silicone emulsion forms between 6.7% and 10% by weight, and preferably 8.37% by weight of the malignant lesion mimicking portion.

16. The tissue mimicking material of claim 14, wherein:
the first portion comprises the fat layer mimicking portion;
the second portion comprises the glandular tissue mimicking portion;
the tissue mimicking material further comprising:
the Cooper Ligament mimicking portion;
the pectoral muscle mimicking portion; and
and any one or more of:
at least one malignant lesion mimicking portion;
at least one benign lesion mimicking portion;
at least one anechoic lesion mimicking portion; and
at least one hyperechoic lesion mimicking portion.

17. A biopsy training device, comprising:
a skin mimicking layer; and
a fibroglandular tissue mimicking portion comprising one or more lesion mimicking portions,
wherein the fibroglandular tissue mimicking portion is removably coupled to the skin mimicking layer.

18. The biopsy training device of claim 17, further comprising;
a base member arranged to removably couple to the skin mimicking layer, wherein the base member and the skin mimicking layer form a housing when coupled in which the fibroglandular tissue mimicking portion is received.

19. The biopsy training device of claim 17, wherein the fibroglandular tissue mimicking portion is coupled to an inside wall of the skin mimicking layer by a coupling liquid comprising water, glycerol and Benzalkonium chloride (BC).

20. A method of manufacturing a tissue mimicking material, comprising the following steps:
a) adding one or more scattering particles to water;
b) adding gelling agent to the mixture formed in step a), wherein the mixture is mixed at a first rate during the addition of the gelling agent;
c) heating the mixture formed in part b), wherein during the heating the mixture is mixed at a second mixing rate, the second mixing rate being slower than the first; and
d) forming the mixture formed in part c) into a desired shape.

21. The method of claim 20, wherein the first mixing rate is between 200 and 260 rpm and the second mixing rate is between 110 and 160 rpm.

22. The method of claim 20, wherein the mixture is at ambient temperature during the addition of the gelling agent in step b).

23. The method of claim 20, wherein the heating of step c) comprises heating to a temperature of 90 to 100° C.

24. The method claim 20, wherein the heating in step c) has a duration of 1 hour.

25. The method of claim 20, further comprising adding glycerol to the resulting mixture formed in step c).

26. The method of claim 25, further comprising heating the glycerol before it is added, the glycerol preferably being heated to a temperature in the range of 70 to 90° C.

27. The method of claim 25, further comprising adding Benzalkonium chloride to the glycerol.

28. The method of claim 25, further comprising adding mould and mildew to the mixture.

29. The method 20, wherein step a) further comprises adding silicone emulsion to the water, the silicone emulsion being added before the addition of the scattering particles.

30. The method claim 20, further comprising adding oil and surfactant to the resulting mixture formed in step c).

31. The method of claim 30, wherein the oil and surfactant is heated before being added, the oil and surfactant preferably being heated to a temperature of greater than 80° C.

32. The method of claim 30, further comprising mixing the mixture resulting from the addition of the oil and surfactant at a third mixing rate.

33. The method of claim 32, wherein the third mixing rate is between 200 rpm and 150 rpm, and preferably comprises a first mixing period of 200 rpm and a second mixing period of 150 rpm.

34. The method claim 30, wherein the mixture is heated during addition of the oil and surfactant and/or during mixing at the third mixing rate.

35. The method claim 32, wherein the mixture is mixed at a fourth mixing rate after mixing at the third mixing rate, wherein the mixture is allowed to cool while mixing at the fourth mixing rate.

36. The method of claim 35, wherein the fourth mixing rate is 250 rpm to 320 rpm and preferably about 290 rpm.

37. The method of claim 20, wherein forming the mixture into the desired shape comprises shaping the mixture in a mould.
